(19) United States
(12) Patent Application Publication
(10) Pub. No.: US 2024/0341899 A1
(43) Pub. Date: Oct. 17, 2024

(54) INFRARED IMAGING FOR DAMAGE DETECTION IN SURGICAL INSTRUMENTS

(71) Applicant: Bedrock Surgical, Inc, Palo Alto, CA (US)

(72) Inventor: Richard Hill, Campbell, CA (US)

(73) Assignee: Bedrock Surgical, Inc, Palo Alto, CA (US)

(21) Appl. No.: 18/682,913

(22) PCT Filed: Aug. 11, 2022
(86) PCT No.: PCT/US22/40133

§ 371 Date: Feb. 11, 2024

Related U.S. Application Data
(60) Provisional application No. 63/232,090, filed on Aug. 11, 2021.

(51) Int. Cl. A61B 90/00; G01N 1/44; G01N 25/72

(52) U.S. Cl. CPC A61B 90/08; G01N 1/44; G01N 25/72; A61B 2090/0809

(57) ABSTRACT

The system and methods described herein are directed to a system for detecting deficiencies in surgical instruments that would justify removing them from service. Traditional visible lighting imaging for detection of small defects is complicated because most instruments are constructed of highly reflective stainless steel and specular reflections can obscure small details such as cracks and pits. Imaging in the infrared spectrum uses the direct emission from the object as opposed to reflected light required for visible imaging and can therefore avoid the issue of specular reflections. Depending on the lighting and orientation of the instrument, small features such as cracks and other damage may be completely obscured and not seen by traditional visible light imaging. Cracks and other damage, such as corrosion and pitting, have been demonstrated to be easily detected using a lab grade camera, although lower cost cameras could be used in a production facility.

### CROSS-REFERENCES

This application claims the benefit of U.S. Provisional Application No. 63/232,090, filed Aug. 11, 2021.

### BACKGROUND

This technology relates generally to imaging of surgical instruments and, more particularly, to methods of detection of abnormalities when the surgical instruments are processed via a consistent repeatable process. U.S. Provisional Application No. 63/232,090, filed Aug. 11, 2021, the entire disclosure of which, except for any definitions, disclaimers, disavowals, and inconsistencies, is incorporated herein by reference.

### SUMMARY

In sterile processing departments, it is of utmost importance that instruments that are being sent to operating rooms are sterile and free of hidden defects. Unfortunately, human visual inspection often misses both physical contamination and instrument damage.

In developing a mechanism to detect issues before they are placed into service it has become clear that there is a need for a repeatable process that allows detection with a high confidence level.

There exists a need for process that consistently catches devices that need to be removed from service.

When deciding about design choices with respect to a system in accordance with the present invention, simplicity is a preferable design choice that is disclosed herein.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of embodiments in accordance with the present invention are apparent in the following detailed description and claims.

### DESCRIPTION OF THE FIGURES

The patent or application file contains at least one image executed in color. Copies of this patent or patent application publication with color drawing(s) will be provided by the Office upon request and payment of the necessary fee.

Various example embodiments can be more completely understood in consideration of the following detailed description in connection with the accompanying drawings, in which:

Various example embodiments can be more completely understood in consideration of the following detailed description in connection with the accompanying drawings, in which:

FIG. 1 shows an infrared image of an instrument with a crack near the hinge point is clearly seen.

FIG. 2 shows linear intensity profile across the crack of the instrument of FIG. 1. The wide intensity peaks are a result of dark ink applied to the instrument for testing purposes. The sharp peak in the center indicates the location of a crack. The location of the linear profile is shown by “Line 1” in the image.

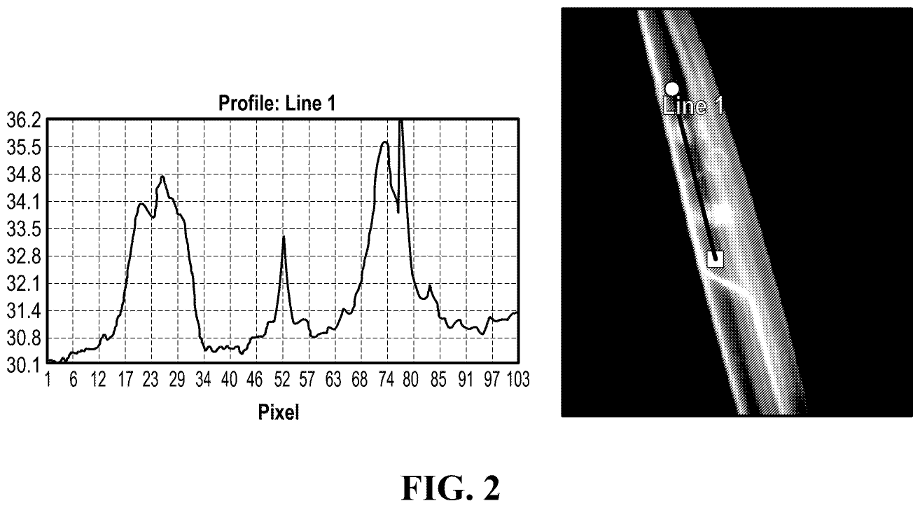

### DETAILED DESCRIPTION

This technology relates generally to surgical instruments and, more particularly, to apparatus' for and methods of assessing if surgical instruments have been damaged.

This disclosure describes inventive concepts with reference to specific examples. However, the intent is to cover all modifications, equivalents, and alternatives of the inventive concepts that are consistent with this disclosure. It will be apparent, however, to one of ordinary skill in the art that the present approach can be practiced without these specific details. Thus, the specific details set forth are merely exemplary, and is not intended to limit what is presently disclosed. The features implemented in one embodiment may be implemented in another embodiment where logically possible. The specific details can be varied from and still be contemplated to be within the spirit and scope of what is being disclosed.

The images below show visible light images of a damaged instrument. The specular reflection from the surface complicates the detection of damage.

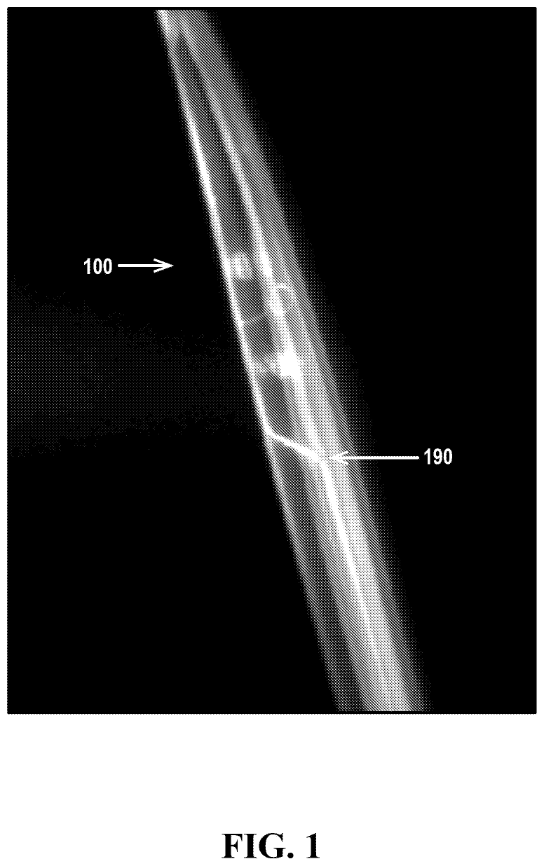

As mentioned above, the problem is generally not addressed until an instrument fails, breaks, or is accidentally detected. Traditional crack detection techniques including dye penetrant approaches are incompatible with sterile instrument processing. Current crack and damage detection techniques, including dye penetrants, are undesirable for use with surgical instruments because they require the application of substances (e.g. dyes) that contaminate the instruments and physical contact with the instruments. Visual observation of cracks 100 (see FIG. 1) and damage, while avoiding contaminants and contact with instruments, is complicated by the reflective nature of the instruments and the resulting specular reflections (see above images).

Very little processing is currently performed on surgical instruments—instruments are washed and sterilized but not routinely inspected for damage including cracks and pitting. Inspection, if performed at all, is a largely manual procedure relying on technicians observing instruments as a secondary task during the cleaning and sterilization process. Automating the inspection process, with the addition of infrared imaging will greatly increases the chances of damage detection.

Cracked or damaged surgical tools must be removed from the inventory before the damage impacts the ability to perform surgery or increases the chances of infection by transferring trapped bioburden/biomass from patient to patient. (Bioburden is defined as viable microorganism present. Biomass is defined as any organic matter from plants or animals.) Often the damaged tool is not detected during preparation and only discovered by the surgeon during surgery, potentially delaying or complicating an operation. Removing damaged devices from the workflow, before being delivered to a surgeon has the potential to greatly increase the efficiency of surgeries and hospitals. Early detection of these issues will alleviate these problems.

Detection of damaged tools as part of the sterile processing workflow would ensure that only safe and fully usable tools are delivered to the operating room, increasing confidence by the surgeon in the tools he or she is provided, increasing productivity by eliminating the potential need for replacement tools, and reducing the chances of infection. Impact would be measured by reduced downtime in the operating room due to less need for instrument replacement as well as reduced incidence of post-surgery infection.

Crack and damage detection in surgical instruments in a sterile processing facility is complicated by the need for a non-contact technique, negating most common techniques. Traditional visible light imaging methods can be difficult because most instruments are made from highly reflective stainless steel and specular reflections obscure small details such as pits and cracks. Imaging in the infrared spectrum however uses direct emission from the object and can avoid the issue of specular reflections. Cracks and other damage, such as corrosion and pitting, have been demonstrated to be easily detected using infrared imaging.

Described herein are systems and methods directed to inspecting instruments using IR imaging. One or more infrared camera or cameras can be used to image devices and visualize cracks and damage that are otherwise difficult to detect. Traditional visible lighting imaging for detection of small defects is complicated because most instruments are constructed of highly reflective stainless steel and specular reflections can obscure small details such as cracks and pits. Imaging in the infrared spectrum uses the direct emission from the object as opposed to reflected light required for visible imaging and can therefore avoid the issue of specular reflections.

All objects emit infrared energy, known as a heat signature. An infrared camera (also known as a thermal imager) detects and measures the infrared energy of objects. The camera converts that infrared data into an electronic image that shows the apparent surface temperature of the object being measured.

Depending on the lighting and orientation of the instrument, small features such as cracks and other damage may be completely obscured and not seen by traditional visible light imaging. Imaging of infrared emission from an instrument presents a solution to these problems. By observing the direct emission in the infrared spectrum (for example the about 8-14 μm thermal band), the specular reflection problem is eliminated, and a clear image of the entire part is observable, allowing observation of cracks and damage for an instrument (see FIG. 1). The image in FIG. 1 was obtained by slightly warming the instrument with a heat gun, to approximately 40° C. and then observing the thermal emission from the instrument with a microbolometer-based long-wave infrared (LWIR) camera. The warming of the instrument could be performed by an external device, such as a heat gun, or could be incorporated into the flow process and take advantage of heating involved in the sterilization procedures.

Further analysis of the image can be used to highlight the cracks and damage. Because cracks are known to occur in the areas around hinges, observation should be focused in those areas. A linear intensity profile is shown in FIG. 3 which highlights the crack. In the profile, which is located along the line shown in the figure on the right, peaks indicated areas of higher emission. These correspond to two broad peaks resulting from dark ink marks applied to the instrument for testing and a sharp mark in the middle indicating the position of the crack near the hinge.

If an instrument is slightly heated (e.g., to 30-40 C), the emitted thermal energy can be imaged using a midwave infrared (MWIR) or long wave infrared (LWIR) camera. Heating could be done using hot air, hot water, or as part of the instrument processing stream (e.g., after instruments are sterilized and still warm). Cracks and other damage, such as corrosion and pitting, have been demonstrated to be easily detected using a lab grade camera, although lower cost cameras could be used in a production facility.

Various imaging arrangements are possible. It is contemplated that the imaging system and methods described above can be incorporated into a movable arrangement for scanning instruments placed in its path. It is also possible for the system and apparatus to be stationary and where the instruments are placed on a movable vessel that brings an array of instruments into the infrared imaging path to be inspected. It is also possible to use the infrared system with a mirror or series of mirrors along with an appropriate detector to interrogate different areas of an instrument surface for defects and/or biomass/bioburden.

The methods described herein can be used in other applications in addition to profiling and inspecting surgical instruments. The inventors have contemplated that the methods described above can be applied to inspecting various other types of instruments for wear and damage.

Other variations and modifications are possible. The description and illustrations are by way of example only. While the description above makes reference to various embodiments, it should be understood that many changes and modifications can be made without departing from the scope of the disclosure. Many more embodiments and implementations are possible within the scope of this invention and will be apparent to those of ordinary skill in the art. It is intended that the appended claims cover such changes and modifications that fall within the spirit, scope and equivalents of the invention. The invention is not to be restricted to the specific details, representative embodiments, and illustrated except in light as necessitated by the accompanying claims and their equivalents.

What is claimed is:

1. A system for surgical instrument defect detection, comprising:
a detection surface, where a surgical instrument is displayed;
an infrared camera detachably coupled with the detection surface, which is used to capture infrared thermal images of the surgical instrument, and to output infrared thermal image digital signals about the inferred thermal images including temperature values of the detected surgical instrument;
a main controller provided on the detection surface and connected to the the infrared camera, which is used to control capture actions of the infrared camera, to transform the infrared thermal image digital signals output from the infrared camera into digital signals to be used in standard network transmission;
a reference database capable of storing reference images of surgical instruments serving as comparators to the detection images generated by the infrared camera of the detected surgical instrument; and
a data processor for generating and outputting control signals for the infrared camera, and to receive the digital infrared signals to be analyzed and processed to determine the types and locations of the defects on the surgical instrument.

2. The apparatus of claim 1, wherein the infrared camera is a mid-wave infrared (MWIR) camera.

3. The apparatus of claim 1, wherein the infrared camera is a long-wave infrared (LWIR) camera.

4. The apparatus of claim 2, wherein the surgical instrument is selected from the group comprising cutting and dissecting surgical instruments; grasping and handling surgical instruments; clamping and occluding surgical instruments; retracting and exposing instruments; instruments for improving visualization; suturing and stapling surgical instruments; and suctioning and aspiration instruments.

5. The apparatus of claim 1, wherein the detection surface has a heating element that allows for the elevation of the surgical instrument temperature to a range about between 25° C.-50° C.

6. A method of detecting surgical instrument defects comprising the steps of:
providing a sample;
providing a sensor:
heating the sample to between 25° C.-50° C.;
observing the thermal emission from the sample with the sensor;
whereby an image is produced free of specular reflection.

7. The method of claim 5, wherein the sample is a surgical instrument.

8. The method of claim 5, wherein the sensor is a mid-wave infrared (MWIR) camera.

9. The method of claim 5, where the sensor is long-wave infrared (LWIR) camera.

10. The method of claim 5, wherein the long-wave infrared (LWIR) Camera is microbolometer-based.
